(19) United States
(12) Patent Application Publication
(10) Pub. No.: US 2005/0215916 A1
(43) Pub. Date: Sep. 29, 2005

(54) Active, multiplexed digital electrodes for EEG, ECG and EMG applications

(76) Inventors: Kalford C. Fadem, Louisville, KY (US); Benjamin A. Schnitz, Brentwood, TN (US)

Correspondence Address: FROST BROWN TODD, LLC, 2200 PNC CENTER, 201 E. FIFTH STREET, CINCINNATI, OH, 45202, US

(21) Appl. No.: 11/092,395

(22) Filed: Mar. 29, 2005

Related U.S. Application Data
(60) Provisional application No. 60/557,230, filed on Mar. 29, 2004.

(51) Int. Cl. A61B 5/04

(52) U.S. Cl. 600/544; 600/546; 600/509

(57) ABSTRACT

A biopotential measurement system incorporates a revolutionary approach to the acquisition of signals such as Electroencephalograms (EEG), Electrocardiograms (ECG), and Electromyograms (EMG) by incorporating active, digital electrodes that amplify and digitally convert biopotential signals at the source, thereby eliminating noise and signal degradation issues. This is to date the most integrated and advanced electrode designed for any biopotential measurement eliminating the poor Signal-to-Noise (SNR) problems seen in biopotential recordings.

### CROSS REFERENCE TO RELATED APPLICATIONS

The present application hereby claims the benefit of the provisional patent application Ser. No. 60/557,230, entitled “ACTIVE, MULTIPLEXED DIGITAL NEURO ELECTRODES FOR EEG, ECG AND EMG APPLICATIONS” filed on 29 Mar. 2004.

### FIELD OF THE INVENTION

The present invention relates, in general, to devices that are attachable to the skin of a patient to detect a biopotential measurement such as an Electroencephalogram (EEG), Electrocardiogram (ECG), and Electromyogram (EMG) electrodes.

### BACKGROUND OF THE INVENTION

The measurement of voltage potentials from the surface of the skin are commonly used to detect a variety of physiological conditions. Voltage potentials generated by the beating heart called ECG's are used to evaluate the performance and condition of the heart and may be indicative of many types of heart disease. EMG's are often detected from electrodes affixed to the skin near muscles to evaluate a subject's neuromuscular activity and may be used to identify muscular dystrophy, peripheral nerve damage or other diseases. EEG's are voltage potentials generated by electrochemical activity within the brain. EEG's are detected by placing electrodes on the scalp and are often used to detect neurological conditions such as epilepsy, schizophrenia, auditory neuropathy, or the effects of anesthesia.

Electroencephalograms (EEGs) have traditionally been the most difficult electrogram measurement to acquire from a hardware standpoint. The signal amplitude for EEGs is tens to hundreds of times smaller than that of ECGs or EMGs. The most commmon EEG application involves using numerous Ag/AgCl electrodes contained within a net or hat placed on the scalp of the patient, with each electrode individually tested for low impedances of less than 10 kΩ. To foster low impedances, technicians often will abrade the scalp of the patient to remove the stratum corneum and use electrolyte gels or saline solutions to couple the electrode to the skin.

The typical EEG net or hat then connects to the hardware box using a cable several feet in length, subjecting the microvolt-level EEG signal to ambient noise that is many times greater than the signal itself. The net effect is that the designer is challenged to extract the very small signal with a poor signal-to-noise ratio in a very narrow frequency range (typically 0.05 to 40 Hz). The design must then incorporate high-order filters with high gain (5000-20000 times) and sharp roll-off to ensure that only the desired signal is recorded for analysis.

These voltage potentials are measured by affixing a plurality of conductive electrodes, at least one of which, the reference electrode, should be placed at a site of minimal electrical activity, and measuring the voltage differential between the reference electrode and the other signal electrodes. The electrodes are commonly made from a conductive material such as silver/silver chloride (Ag/AgCl) or gold (Au) and are often wetted with a conduction enhancing solution such as saline or a conductive gel.

The voltage differential between the reference electrode and the signal electrodes is extremely small, on the order of millivolts (10-3 mV) or microvolts (10-6 μV). To detect the small physiological signal in the presence of background electrical noise requires amplification and filtering. The amplification and filtering is usually accomplished via an amplifier box connected to the electrodes with long wires.

The amount of signal amplification and the settings of the filters must often be adjusted based on the biopotential signal being measured. This function is usually performed by potentiometers and adjustable filters within the amplifier box.

The signal from the amplifier box is often converted to a digital format, in order to store the signals on a computer or to perform modern digital signal processing functions such as using the Discrete Fourier Transform for spectral analysis. The analog to digital (A/D) conversion is usually performed by specialized hardware within the amplifier box or within a separate A/D converter box. If multiple signal channels are used, there is typically a discrete A/D converter circuit for each channel.

For many biopotential measurement applications, the long electrode wires which transmit the raw signal from the electrode to the amplifier box present a number of problems, both in terms of the utility of the system and the accuracy of the measurements. This is for a number of reasons. First, the wires act as an antenna which will pick up stray background electrical noise, which could come from other powered equipment such as electrosurgical devices used to cauterize wounds. Electrical filters in the amplifier box are used to limit the degradation caused by background noise but in doing so, the filters also mask or modify a certain amount of the signal. The second reason that long wires limit the accuracy of the detected signals is that the signals are very small and consequently, there is a certain amount of signal loss due to the impedance of the wire.

It would be desirable to perform variable gain signal amplification, filtering, and A/D conversion as close to the electrode contact point as possible. Therefore, the signal could be amplified, filtered, and converted to a digital format with a minimum of signal degradation and induced noise.

BioSemi markets a preamplified electrode for biopotential measurements. With this system, BioSemi has developed an electrode contact with integrated amplifiers. This system uses a fixed value amplifier to the contact point. The signals are then sent along a wire to a junction box where the signal is amplified again and then converted to a digital signal. While this system amplifies the signal close to the electrode, the long analog signal wires between the electrode and the junction box are still problematic. This system also requires an additional amplification step before the signal is digitized so that any noise picked up from the long wire will be included in the digitized signal.

Thought Technology LTD markets a variety of biopotential electrodes: MyoScan-Pro, MyoScan, and EEG-Z. These preamplified electrodes can be attached to an integrated electrode strip. This system, like the BioSemi system, uses a fixed value amplifier close to the electrode contact but uses long electrode wires to send the analog signal to an interface box for conversion to digital format.

Consequently, a significant need exists for an electrode device suitable for clinical use that achieves improvements in signal-to-noise ratio for weak biopotential measurements.

### BRIEF SUMMARY OF THE INVENTION

The invention overcomes the above-noted and other deficiencies of the prior art by providing active filtering and digitization of sensed biopotential measurements in a circuit that is in close proximity to the patient's skin. The digitized signals are then multiplexed across an electromagnetic channel (e.g., IR or RF broadcast, electrically conducted, optically guided) to a remote controller that selects which electrode to sample. The selection rate and other control criteria to the respective electrods may advantageously be selected to correspond to the active filtering required for a type of biopotential (e.g., Electroencephalogram (EEG), Electrocardiogram (ECG), and Electromyogram (EMG)).

In one aspect of the invention, an apparatus for sensing a plurality of biopotential voltages on a subject with a reference electrode attachable to skin of the subject is used to differentially sense to a first and second digital electrodes attachable to the skin of the subject. Each digital electrode has at least one conductive contact coupled to an active frequency filter responsive to a differential input of the reference electrode and the respective analog electrode. An analog-to-digital converter that is coupled to an amplified filtered output of the active frequency filter produces a respective amplified digital signal that is multiplexed across an electromagnetic channel to a controller in two-way communication over the external electromagnetic channel that sequentially selects the respective amplified digital signal from the first and second digital electrodes. Thereby, the electromagnetic channel is reduced to a small number of signals that may be conveniently provided to a patient without elaborate conduits and supports.

In another aspect of the invention, an active electrode improves a signal-to-noise ratio for sensing a biopotential signal (e.g., EEG, EMG, ECG) by isolating a ground plane of an analog portion of an electrode circuit from a ground plane of a digital portion of thereof to prevent ground loops wherein digital noise is prevented, or at least greatly reduced, from distorting the weak input signals, especially EEG. Thereby, full functionality may be incorporated into close proximity with the electrode contact to the skin.

In yet another aspect of the invention, an active electrode improves a signal-to-noise ratio of a sensed biopotential signal by setting a variable gain of a filtered analog biopotential signal prior to analog-to-digital conversion so as to take full advantage of the resolution of the converter. Thus, even with widely varying skin impedances and thus strength of biopotential, the active electrode achieves a filtered analog signal for digital conversion that does not saturate the converter nor is so small as to make the resolution limit of the converter be a significant contributor to signal-to-noise ratio degradation.

These and other objects and advantages of the present invention shall be made apparent from the accompanying drawings and the description thereof.

### DESCRIPTION OF THE FIGURES

The accompanying drawings, which are incorporated in and constitute a part of this specification, illustrate embodiments of the invention, and, together with the general description of the invention given above, and the detailed description of the embodiments given below, serve to explain the principles of the present invention.

FIG. 1 is a block diagram of a biopotential measurement system with active electrodes that enable multiplexed, digital measurements to a control box.

FIG. 2 is a block diagram of one of the active electrodes of FIG. 1.

FIG. 3 is an electronic circuit schematic of an illustrative active electrode of FIG. 2.

FIGS. 4A-4B are a diagram of top and bottom surfaces of a printed circuit board layout of the active electrode of FIG. 3.

FIG. 5 is a bode plot of the frequency response of the active electrode of FIG. 3.

FIG. 6 is a top view of a flexible printed circuit for a multi-electrode device for the biopotential measurement system of FIG. 1.

FIG. 7 is a perspective exploded view of an active electrode of FIG. 3.

### DETAILED DESCRIPTION OF THE INVENTION

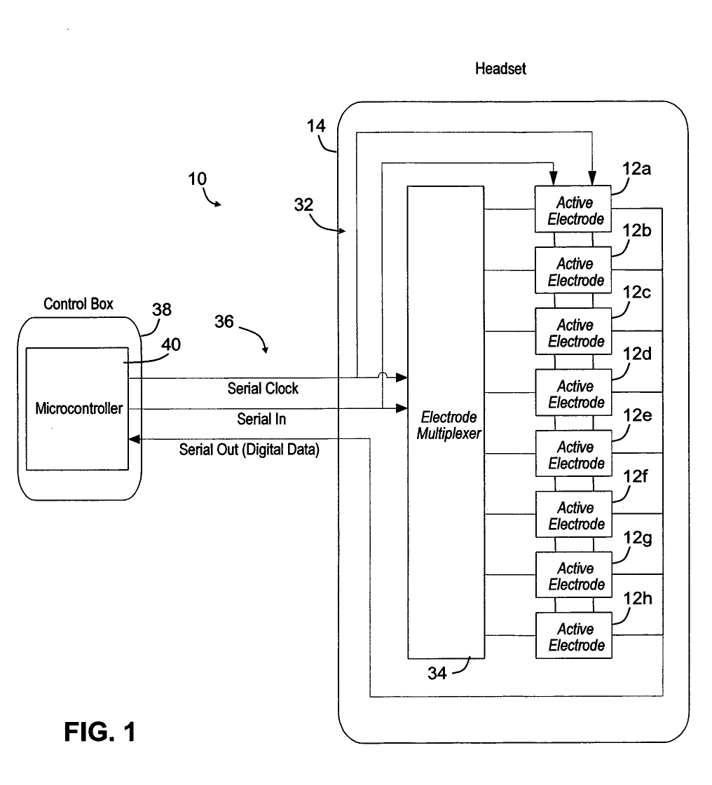

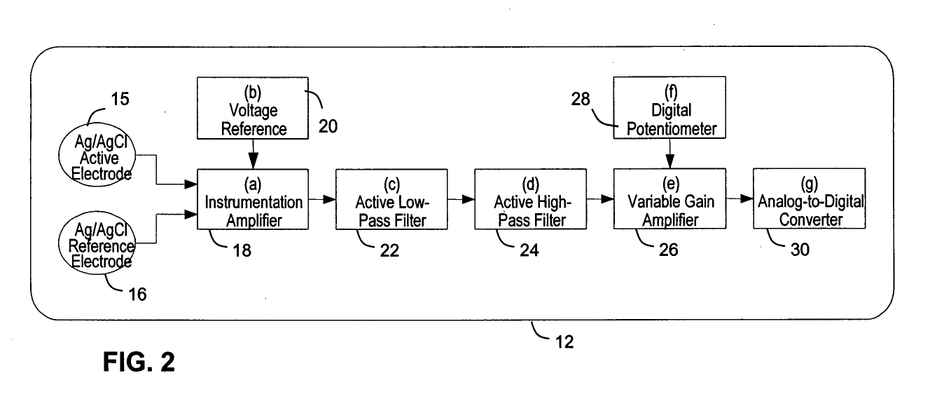

In FIGS. 1-2, a biopotential measurement system 10 is designed using a revolutionary approach to the acquisition of an electroencephalogram (EEG) by incorporating active, digital electrodes 12a-12h into a headset 14 that amplifies and digitally converts an EEG signal at the source, thereby eliminating noise and signal degradation issues. This is to date the most integrated and advanced electrode designed for any electrogram measurement. To significantly reduce the poor Signal-to-Noise (SNR) problems seen in EEG recordings, amplification and filtering electronics are incorporated into each electrode.

With particular reference to FIG. 2, each active, digital electrode 12 senses an electrogram as a biopotential differentially between a respective AG/AgCl signal conductive contact 15 and a shared Ag/AgCl reference conductive contact 16 is first amplified using an instrumentation amplifier 18 with a fixed output gain of 50 referenced to a voltage reference 20 and a very high input impedance (>100 G∩) to reduce effects of high skin-electrode impedance. The voltage reference 20 is formed from an operational amplifier configured as a buffer to provide a low-impedance reference that is set using an input voltage that is divided to half of the supply voltage.

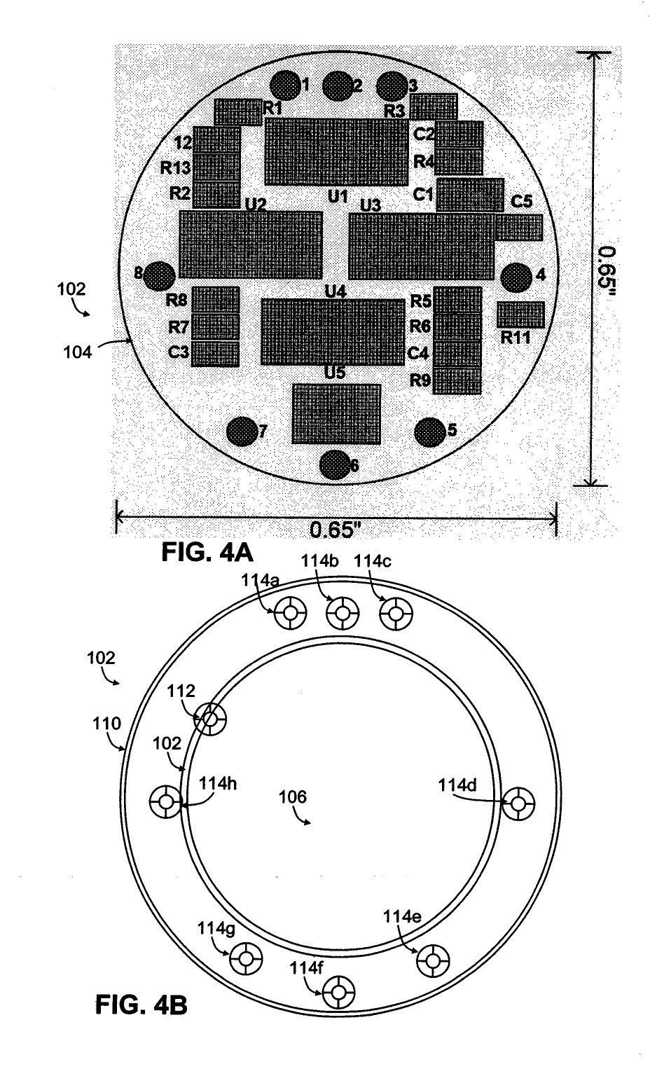

Next, the signal is filtered using a 2nd-order Butterworth low-pass filter 22 set at 30 Hz with a filter gain of 10, 3 db ripple pass band. The low pass filter 22 is followed by a first-order active high-pass filter 24 to eliminate electrode offset potentials. The active high pass filter 24 is formed from an operational amplifier configured to have a 0.1 Hz cutoff and buffered to provide sharper roll-off. The signal then is amplified by a variable amount by a variable gain amplifier 26, which in the illustrative version is an active inverter with a low-pass cutoff of 30 Hz formed from an operational amplifier with a fixed feedback resistor and a variable feedback resistor. In particular, the variable gain is set by a variable feedback resistance provided by a digital potentiometer 28 which is located on an electrode printed circuit board (PCB) (FIGS. 4A-4B). The variable amplification of 50-10,000 thus allows total system gain of (50*10*50)−(50*10*10,000)=2,500-500,000.

These active electrodes 12a-12h are also the first such electrodes to contain high-resolution A/D conversion on board, so that the only output from each electrode is a digitized signal at a point physically located less than 15 mm from the Ag/AgCl signal conductive contact 15 itself. Each active electrode 12 contains a single 16-bit Analog-to-Digital (A/D) converter 30, which in the illustrative version is a 16-bit Successive Approximation Register (SAR) architecture A/D converter.

With particular reference to FIG. 1, each active electrode 12a-12h operates on a Serial Peripheral (SPI) bus 32, allowing individual electrodes to be “activated” using a chip select function selected through an electrode multiplexer 34 also on the SPI bus 32. This allows all electrodes 12a-12h to share a single digital output connection and reduces the number of wires in a conduit 36 (e.g., wire bundle) between the headset 14 and a control box 38 that contains a microcontroller 40.

It should be appreciated that the conduit 36 may comprise an electromagnetic channel wherein the two-way communication is formed by broadcast signals, electrically conducted signals, or a fiber optic guided signal.

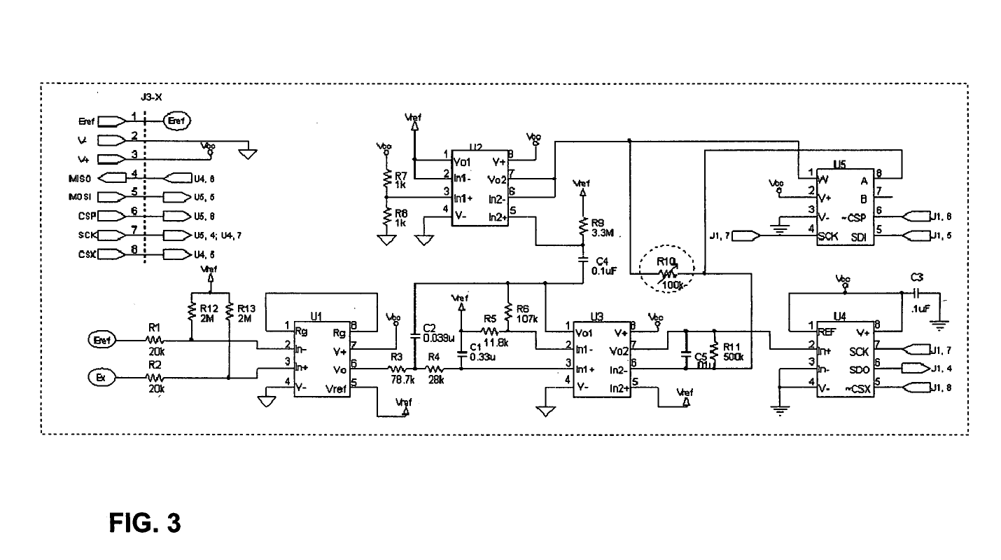

In FIGS. 3-4A, 4B, an illustrative active electrode circuit 100 is depicted for performing the signal processing described above for the active electrode 12 of FIG. 2. Each electrode 12a-12h is a printed circuit board (PCB) 102 containing surface-mount electronics that facilitate the amplification, filtering, and digital conversion of a single electrogram. The PCB 102 includes an Integrated Circuit (IC) Single-Supply, Rail-to-Rail Output, Complementary Metal-Oxide Semiconductor (CMOS) instrumentation amplifier (“U1”) 101 in 8-mini small outline package (MSOP), By Texas Instruments, Part No. INA155E/250 that is a principal component of the instrumentation amplifier 18. Two IC Single Supply CMOS Operational Amplifiers (“U2, U3”) in 8-MSOP, By Texas Instruments, Part No. OPA2335AIDGKT provide the op amps for the voltage reference 20, low pass filter 22, high pass filter 24 and variable gain amplifier 26. An IC 16-Bit, High-Speed, unipolar serial analog-to-digital converter U4 in 8-MSOP, By Texas Instruments, Part No. ADS8320E/250 performs the conversion of the A/D converter 30 with gain controlled by an IC 256-Position SPI Compatible Digital Potentiometer U5, Analog Devices, Part No. AD5160BRJ50-R2 that acts as digital potentiometer 28.

A bus connector J3-X, which in the illustrative version is a 9 position male circular connector plug by HIROSE, Part. No. HR25-9P-12P connects the active electrode circuit 100 to other components over serial peripheral bus 32. In particular, the Ag/AgCl reference conductive contact 16 is connected to Pin 1. Pin 2 (V−) is connected to a circuit return of active electrode circuit 100. Pin 3 (V+) is connected to voltage common collector Vcc. Pin 4 is connected between Pin 6 of the A/D converter U4 to provide bus signal MISO (Master-In-Slave-Out). Pin 5 receives bus signal MOSI and provides it to digital potentiometer U5. Pin 6 receives bus signal CSP and provides it to pin 5 of the digital potentiometer U5. Bus signal CSP is received at pin 6 and is provided to Pin 6 of the digital potentiometer U5. Bus signal SCK is received at Pin 7 and is provided to Pin 4 of the digital potentiometer U5 and to Pin 7 of the A/D converter U4. The bus signal CSX is received at Pin 8 and is provided to Pin 5 of the dual op amp U4.

With particular reference to FIG. 3, active electrode circuit 100 includes configuring the instrumentation amplifier U1 as follows. Pin 1 (Rg) is connected to Pin 8 (Rg). Pin 2 (In−) is connected to Ag/AgCl reference conductive contact 16 by 20 kΩ Resistor R1 and to Vref by 2 M1 Resistor R12. Pin 3 is connected to Ag/AgCl signal conductive contact 15 by 20 kΩ Resistor R2 and to Vref by 2 MΩ Resistor R13. Pin 4 (V−) is connected to a circuit return. Pin 5 (Vref) is connected to Vref. Pin 6 (Vo) is connected to a Pin 3 (In1+) of dual op amp IC U3 via a series of 78.7 kΩ Resistor R3 and 28 kΩ Resistor R4. At the junction of Resistors R3, R4, a series of a 0.039 μF Capacitor C2 and a 0.1 μF Capacitor to Pin 5 (In2+) of the dual op amp IC U2. Pin 7 (V+) is connected to voltage common collector (Vcc).

The dual op amp IC U2 is further configured as follows. Pin 1 (Vo1) and Pin 2 (In1−) are connected to Vref. Pin 3 (In1+) is connected to VCC via a 1 kΩ Resistor R7 and to circuit return by a 1 kΩ Resistor R8. Pin 4 (V−) is connected to the circuit return. Pin 5 (In2+) is also connected to Vref via 3.3 MΩ Resistor R9. Pin 6 (In2−) and Pin 7 (Vo2) are both connected to Pin 1 (W) of digital potentiometer IC U5. Pin 8 (V+) is connected to Vcc.

The dual op amp IC U3 is further configured as follows. Pin 1 (Vo1) is connected to the junction between Capacitors C2, C4. Pin 2 (In1−) is connected to Pin 1 via 107 kΩ Resistor R6 and to Vref via 11.8 kΩ Resistor R5. Pin 3 (In1+) is further connected to Vref via 0.33 μF Capacitor C1. Pin 4 (V−) is connected to the circuit return. Pin 5 (In2+) is connected to Vref. Pin 6 (In2−) is connected to Pin 7 (Vo2) via a parallel combination of a 0.1 μF Capacitor C5 and 500 kΩ Resistor R11 and to Pin 8 (A) of digital potentiometer U5. Pin 7 (Vo2) is also connected to Pin 2 (In+) of A/D converter IC U4. Pin 8 is connected to Vcc.

The digital potentiometer IC U5 is further configured as follows. Pin 1 (W) is connected to Pin 8 (A) by O-to-50 kΩ tunable resistor R10. Pin 2 (V+) is connected to Vcc. Pin 3 (V−) is connected to the circuit return. Pin 7 (B) is unused.

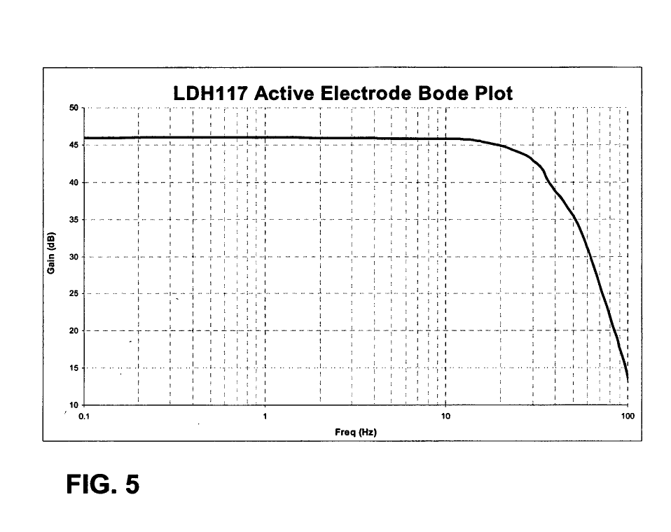

In FIG. 5, the electronic circuit 100 of FIG. 3 achieves a desired level pass band response for frequencies of interest of 30 Hz or lower.

In FIGS. 4A-4B, the PCB 102 advantageously includes a physical layout of the active circuitry on a top surface 104 (FIG. 4A) that puts the active components into close proximity to the conductive contact (Ag/AgCl), whose attachment surface 106 is depicted in FIG. 4B on a bottom surface 110 of the PCB 102. A drill hole 112 depicted in FIG. 4B corresponds to the pass-through of the biopotential signal from the bottom surface 110 to the top surface 104. Eight other small holes 114a-114h visible on the bottom surface 110 are attachment points for databus and power supply wires on the top surface 104.

It should be appreciated that the printed conductive traces are advantageously small on the top surface 104 so as to avoid presenting an antenna at the frequency ranges of interest to reduce electromagnetic interference. Further, analog portions of the circuit (i.e., all but variable gain amplification and analog-to-digital conversion) have an analog ground path tied to a common electrical node that is attached to an external ground conductor. The digital portions of the circuit have a separate ground path that only connect to the analog ground path at the common electrical node.

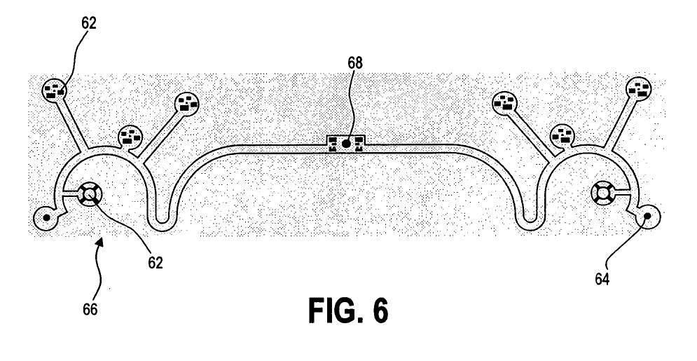

In FIG. 6, an alternative interconnection of active electrodes 62 and reference electrodes 64 on a flexible printed circuit 66 is advantageously shaped to position these electrodes 62, 64 at predetermined locations on the subject's body (e.g., cranium) with a serial bus port connector 68 also at a convenient location. Moreover, printing the conductive traces with conductive inks results in an economical device.

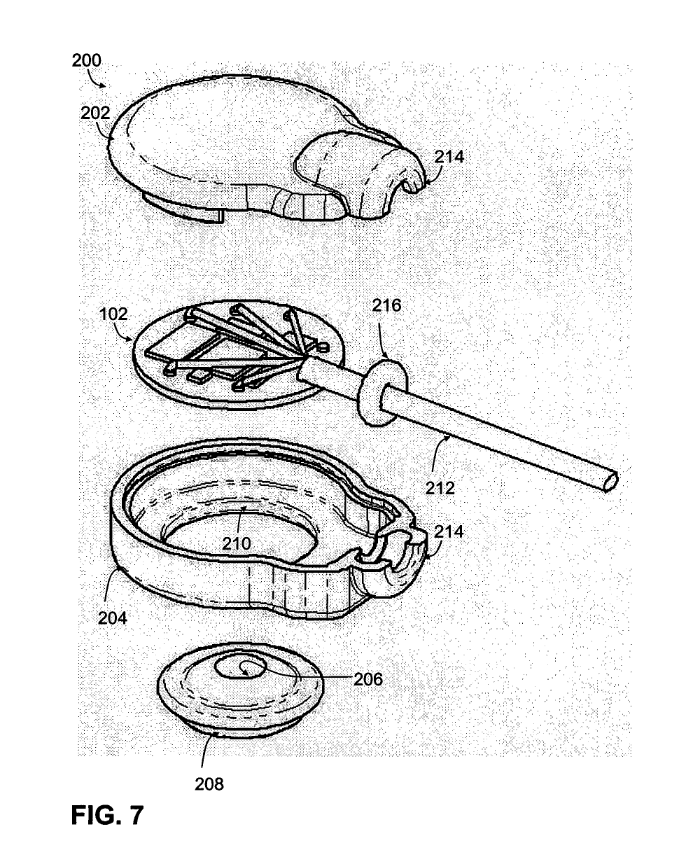

In FIG. 7, an active electrode 200 is depicted incorporating the PCB 102 captured between an electrode upper housing 202 and an electrode lower housing 204. Electrodes (not shown) on an undersurface of the PCB 102 are exposed to the skin through a central aperture 206 in a resilient suction cup 208 exposed through a lower aperture 210 in the electrode lower housing 204. An electrical cable 212 passes through a port 214 formed between the upper and lower housings 202, 204 and is provided with a strain relief resilient disk 216 encompassing the cable 212 trapped in the port 214.

While the present invention has been illustrated by description of several embodiments and while the illustrative embodiments have been described in considerable detail, it is not the intention of the applicant to restrict or in any way limit the scope of the appended claims to such detail. Additional advantages and modifications may readily appear to those skilled in the art.

1. An apparatus for sensing a plurality of biopotential voltages on a subject, comprising:
a reference conductive contact attachable to the skin of the subject,
first and second digital electrodes attachable to the skin of the subject, each digital electrode comprising at least one signal conductive contact coupled to an active frequency filter, coupled responsive to a differential input of the reference conductive contact and the respective signal conductive contact, and an analog-to-digital converter coupled to an amplified filtered output of the active frequency filter to produce a respective amplified digital signal;
an electromagnetic channel;
a controller in two-way communication over the external electromagnetic channel and operatively configured to sequentially select the respective amplified digital signal from the first and second digital electrodes; and
a multiplexer communicating between each digital electrode and the electromagnetic channel responsive to the controller to communicate a digital output from the selected digital electrode.

2. The apparatus of claim 1, wherein the first and second digital electrode each further comprise a respective analog ground plane electrically connected to a common electrical node to prevent electrical ground loops.

3. The apparatus of claim 1, wherein the active frequency filter further comprises a buffer filter isolating a ground plane of the active frequency filter and analog-to-digital converter from the analog electrodes.

4. The apparatus of claim 3, wherein the active frequency filter further comprises a variable gain amplifier responsive to the controller to set an amplification of the amplified filtered output.

5. The apparatus of claim 4, wherein the active frequency filter further comprises an instrumentation amplifier responsive to a respective analog electrode, the reference electrode and a voltage reference to produce a sensed biopotential signal; an amplifier filter operatively configured to produce a frequency band limited, amplified analog biopotential signal for the variable gain amplifier.

6. The apparatus of claim 1, wherein the electromagnetic channel comprises a digital databus including a clock signal to the multiplexer to select one of the first and second digital electrodes and a serial out signal from the multiplexer carrying the digital output from the selected digital electrode.

7. The apparatus of claim 1, wherein the active frequency filter further comprises a variable gain amplifier responsive to the controller to set an amplification of the amplified filtered output to the analog-to-digital converter, the electromagnetic channel including a serial in signal from the controller setting the amplification.

8. The apparatus of claim 1, wherein the active frequency filter is operatively configured to sense and amplify an electroencephalogram (EEG) signal from the analog electrode.

9. The apparatus of claim 1, wherein the active frequency filter is operatively configured to sense and amplify an electrocardiogram (ECG) signal from the analog electrode.

10. The apparatus of claim 1, wherein the active frequency filter is operatively configured to sense and amplify an electromyogram (EMG) signal from the analog electrode.

11. The apparatus of claim 1, further comprising a flexible printed circuit shaped to position the reference conductive contact and the first and second digital electrodes as predetermined locations on the subject and comprising printed conductive traces supporting electronic components as each location that comprise respective first and second active electrodes.

12. A device for sensing a biopotential voltage on a subject, comprising:
a reference conductive contact attachable to the skin of the subject;
a signal conductive contact attachable to the skin of the subject;
an instrumentation amplifier operatively configured to sense a differential analog signal across the reference and signal conductive contacts;
a bandpass filter operatively configured to filter the sensed differential analog signal; and
an analog-to-digital converter in communication with the bandpass filter to produce a digital biopotential signal;
a circuit board including an external ground attachment point and supporting the signal conductive contact on an undersurface and supporting the instrumentation amplifier, bandpass filter, and analog-to-digital converter on a top surface, wherein ground loops allowing downstream digital noise to be amplified in upstream analog components are prevented by segregating a ground return from the analog-to-digital converter

13. The device of claim 12, further comprising a variable gain amplifier interposed between the bandpass filter and the analog-to-digital converter, the variable gain amplifier sharing the ground return with the analog-to-digital converter.

14. A device for sensing a biopotential voltage on a subject, comprising:
a reference conductive contact attachable to the skin of the subject;
a signal conductive contact attachable to the skin of the subject;
an instrumentation amplifier operatively configured to sense a differential analog signal across the reference and signal conductive contacts;
a variable amplifier operatively configured to respond to a command to set a variable gain of the sensed differential analog signal; and
an analog-to-digital converter in communication with the variable amplifier to produce a digital biopotential voltage scaled to the variable gain.
